(19) United States
(12) Patent Application Publication
(10) Pub. No.: US 2024/0314502 A1
(43) Pub. Date: Sep. 19, 2024

(54) BIOMIMETIC MICROPHONE AND COCHLEAR IMPLANT COMPRISING SAID BIOMIMETIC MICROPHONE

(71) Applicant: Stichting Radboud Universiteit, Nijmegen (NL)

(72) Inventor: Martijn AGTERBERG, Nijmegen (NL)

(73) Assignee: Stichting Radboud Universiteit, Nijmegen (NL)

(21) Appl. No.: 18/577,135

(22) PCT Filed: Jul. 6, 2022
(86) PCT No.: PCT/EP2022/068771

§ 371 Date: Jan. 5, 2024

(30) Foreign Application Priority Data
Jul. 6, 2021 (NL) 2028629

(51) Int. Cl. H04R 25/00; A61N 1/36

(52) U.S. Cl. CPC H04R 25/402; A61N 1/36038; A61N 1/36128

(57) ABSTRACT

The invention relates to a biomimetic microphone, a product comprising at least one biomimetic microphone, such as a hearing implant, wherein the hearing implant may comprise a cochlear implant, or a vibrating implant, or both, a method of operating a hearing implant, and a hearing implant computer program comprising instructions for operating the hearing implant.

### FIELD OF THE INVENTION

The invention relates to a biomimetic microphone, a product comprising at least one biomimetic microphone, such as a hearing implant, wherein the hearing implant may comprise a cochlear implant, or a vibrating implant, or both, a method of operating a hearing implant, and a hearing implant computer program comprising instructions for operating the hearing implant.

### BACKGROUND OF THE INVENTION

The ear enables hearing and, in mammals, balance. In mammals the ear may be described as having three parts, namely the outer ear, the middle ear and the inner ear. The present invention is focused on the inner ear, in particular on the cochlea and hair cells in the cochlea. The inner ear is located in a bony labyrinth, and contains structures which are considered to be essential to several senses: the semi-circular canals, which enable balance and eye tracking when moving: the utricle and saccule, which enable balance when stationary; and the cochlea, which enables hearing. As the present invention primarily relates to hearing, the cochlea is in that respect of most interest.

The cochlea (Greek for “snail”) is a tonotopically organised spiral-shaped, hollow, conical chamber of bone, in which sound waves propagate. The cochlea includes three scalae or chambers, namely a vestibular, which lies superior to the cochlear duct and abuts the oval window, a tympanic duct, which lies inferior to the cochlear duct and terminates at the round window, and a cochlear duct that the stereocilia of the hair cells project into. Further the cochlea includes the helicotrema, Reissner's membrane, the osseous spiral lamina, the basilar membrane. Corti's organ, the sensory epithelium, a cellular layer on the basilar membrane, in which sensory hair cells are located, and the spiral ligament. The tonotopic organization of the hair cells means that each position on the basilar membrane represents a certain frequency. Lower frequencies have waves that propagate along the complete cochlea, whereas higher frequencies propagate less far. In humans frequencies from 100 Hz up to 20 kHz are represented. The tonotopic organization that has its foundation in the cochlea is maintained in the entire auditory system. In the brainstem up to the auditory cortex a specific location in the brain represents a certain frequency. So, in contrast to the visual system the auditory system is not organized in a spatial manner. Spatial information is processed by integration of signals originating from the two cochleae. The cochlea receives sound in the form of sound vibrations, which cause the stereocilia to move. The stereocilia then convert these vibrations into nerve impulses which are taken up to the brain to be interpreted by the brain, that is, when sound is perceived. Two fluid-filled outer spaces are present as well. Air or fluid in general is not well compressible, and therefore the fluid volume needs to exit somewhere.

In the cochlea, hair cells are arranged in four rows in the organ of Corti along the entire length of the cochlear coil. The inner hair cells provide the main neural output of the cochlea towards to the auditory nerve. The outer hair cells mainly receive neural input from the brain, which influences their motility as part of the cochlea's mechanical pre-amplifier. The input to the outer hair cells is from the olivary body via the medial olivocochlear bundle. The cochlear duct has a complex shape. The cochlea is filled with a watery liquid, which moves in response to the vibrations coming from the middle ear via the oval window. As the fluid moves, thousands of hair cells sense the motion, and convert that motion to electrical signals that are communicated via neurotransmitters to many thousands of nerve cells. These primary auditory neurons transform the signals into electrochemical impulses known as action potentials, which travel along the auditory nerve to structures in the brainstem for further processing. When signals are receiving the auditory cortex subjects will can a conscious percept of sound (location). The hair cells are adapted to receive limited sound frequencies by their location in the cochlea (tonotopic organization), and due to varying stiffness of the cells. Ears need to be protected from noise, such as loud noise, continued noise, etc. Noise may cause hair cells to die, eventually. This is a common cause of partial hearing loss.

The present invention is amongst others in the field of a hearing implant, and in particular vibrating hearing implants like bone-conduction implants and middle ear implants, but also cochlear implants. Vibrating hearing implants are provided to persons with a conductive hearing loss. A cochlear implant is typically a surgically implanted neuroprosthesis that provides a person with sensorineural hearing loss a modified sense of sound. The implant bypasses the normal acoustic hearing process. It provides electric signals which directly stimulate the auditory nerve. A person with a cochlear implant may learn to interpret those signals as sound and speech, especially when hearing ability was present, and was lost relatively shortly ago. Otherwise intensive auditory training may be required, which is far more cumbersome.

The cochlear implant typically has two main components. An outside component, which is generally worn on the skin of the head and coupled with a magnet. The outside component typically comprises a sound processor, comprising microphones, electronics, for signal processing, and typically digital signal processing, a battery, and a transmitter, such as a coil, that transmits a signal to the inside component across the skin. The inside component, the actual implant, likewise has a receiver, such as a coil, to receive signals, and often further electronics, and an array of electrodes which is placed into the cochlea, which stimulates the cochlear nerve. Surgical risks of implantation are considered minimal.

From the early days of implants speech perception via an implant has steadily improved. Many users of modern implants obtain reasonable to good hearing and speech perception skills after implantation. One of the challenges that remain with these implants is that hearing and speech understanding skills after implantation show a wide range of variation across individual implant users and speech understanding in noisy conditions is in general poor. Factors such as duration and cause of hearing loss, how the implant is situated in the cochlea, the overall health of the cochlear nerve, but also individual capabilities of re-learning are considered to contribute to this variation, yet no certain predictive factors are known. A further issue is that typically hearing implants are provided to both ears/cochleae. These two implants provide unreliable information in terms of interaural time and level differences. Signals from hearing implants do not fuse sufficiently, such as at the level of the brainstem. As a consequence of the inappropriate integration of the information ascending from the auditory nerves, no accurate (enough) binaural processing in the brain area is possible. Therefore, bilateral application of hearing implants results mainly in the ability to lateralize sounds and not in the ability to indicate the precise sound location. Consequently, sound localization in noisy backgrounds is also not optimal. Beam-formers in microphones for hearing implants are considered to be not accurate enough and processing is poor in terms of spatial and frequency resolution.

Some documents may be referred to. US 2021/096208 A1 recites an apparatus comprising at least one first microphone which is movably arranged, at least one second stationary microphone and at least one sensor is described. The microphones can capture the sound waves emitted by acoustic sources, and the sensor can capture spatial coordinates of the first microphone. A corresponding method and a system having the apparatus mentioned are also described. EP 2 449 795 A1 recites a method including: obtaining phase information dependent upon a time-varying phase difference between captured audio channels: obtaining sampling information relating to time-varying spatial sampling of the captured audio channels; and processing the phase information and the sampling information to determine audio control information for controlling spatial rendering of the captured audio channels. US 2020/236475 A1 recites a hearing aid is provided for use with a user having a first and second ears disposed on first and second body sides. The hearing aid apparatus is configured for enabling the user to hear sounds that originate from a plurality of directions and includes a first hearing aid member placeable on a user's first body side. The first hearing aid member includes a first transducer for receiving sounds that would be received by the user's first ear and converting those received sounds into first transmittable electrical signals. A second hearing aid member is placeable on the user's second body side and is preferably a cochlear implant device including an electrode array positionable within a cochlea of a user. The cochlear implant device includes a second transducer for receiving sounds that would be received by the user's second ear, and converting the sounds into second electrical signals; and also includes a receiver for receiving the first transmittable electrical signals, and a first signal processor for processing the second electrical signals and first transmittable electrical signals into signals configured for being received by the cochlea of user's second ear for facilitating the hearing of sounds that would be received by both of the user's first and second ears.

It is an object of the present invention to overcome one or more disadvantages of the microphones and hearing implants of the prior art and to provide alternatives to current implants, without jeopardizing functionality and advantages.

### SUMMARY OF THE INVENTION

It has now been found that the present biomimetic microphone results in a (nearly) perfect beam-former. In an example the problem of lack of accuracy of the prior art is solved by moving at least one of the microphones. Instead of sending unreliable information to the brain over the two auditory nerves, the present biomimetic microphone, in combination with an implant, sends reliable and systematic information to the brain over one auditory nerve only. In an example a pulsating microphone, a rotating microphone, a dynamic microphone, or a combination thereof, can be used. Especially the inclusion of a dynamic microphone, in particular in an outside part of a hearing implant, is considered novel and inventive. In the prior art technology in hearing implants microphones are static. A novel aspect is to include at least one dynamic microphone, such as one that rotates or pulsates. A disadvantage may be the battery consumption. However it is relatively easy to overcome this minor disadvantage, as charging of a battery nowadays typically does not form an obstacle to any use of an electronic device. The at least one dynamic microphone, typically in combination with at least one static microphone, provides spatial localization of sound. The present biomimetic microphone comprises at least two audio receivers, such as two microphones. The biomimetic microphone, as well as input provided thereto and output provided thereby, may be controlled in an analogue manner, or in a digital manner, or a combination thereof. An at least one first audio receiver in particular being at a first position in the biomimetic microphone, typically in a static position, and at least one second audio receiver at a distance from the first position, wherein the at least one second audio receiver is adapted to receive sound in a plane in at least one sequence, in particular in a cyclic sequence, wherein the at least one sequence is continuous or discrete, wherein the plane is selected from a circle, an ellipsoid, a surface section of a sphere, such as a concave or convex section of a sphere, a surface section of a cone, or a surface section of a cylinder, which at least one second audio receiver may be considered as a dynamic audio receiver, in that a position of reception of sound varies in time, in which, in a particular example, the first audio receiver may form an element of the second audio receiver. The term “sequence” is used in its normal meaning, namely a series [of receptions] in which repetitions are allowed and order matters. The elements of the sequence are typically obtained with a separation in time between elements. As the at least one second audio receiver is adapted to receive sound in a plane the sequence is both spatially resolved and time resolved, either discrete or (semi-)continuous. Also the term “cyclic” is used in its normal meaning [arranged in or belonging to a cycle], namely a series of related events or operations happening regularly and usually leading back to a starting point thereof, so beginning at some point in time or space, moving forward in time or space, and returning to the initial or first point in time or space. It is noted that the plane can be curved, such as in the examples given. The at least one first audio receiver and at least one second audio receiver are spaced apart. It is an option to place the at least one first and at least one second audio receivers relatively far apart, such as with the head in between the audio receivers, or to use analogue processing (no time delay). Placing the at least one first audio receiver and at least one second audio receiver in one housing, or one device, is probably a second-best solution. An optimal solution could be to provide the brain with accurate binaural cues. Unfortunately, all the work in this area the last decade did not result in a practical solution. The present solution can be standardised. The combination of the at least one first audio receiver and the at least one second audio receiver is therefore adapted to receive spatial audio input, that is a resolution of a spatial direction of a perceived sound source, or multiple sound sources for that matter, is possible. Thereto at least one processor for processing audio input, and for providing output, is provided, which at least one processor may have further functionality. The at least one processor for processing audio input of the at least two audio receivers is in particular configured to process the input using at least one of Fourier transforming the audio input, inverse-Fourier transforming the transformed audio input, reducing white noise, filtering white noise, reducing background noise, filtering background noise, using a directional sensitive filter, using a bandpass filter, more in particular a filter with a bandwidth from 350 Hz-17 KHz, even more in particular a bandwidth from 900 Hz-6 kHz, and then providing output. Also, in use, a power source, such as a battery, is typically present. The present biomimetic microphone can find application in for instance a cochlear implant, in auditory research, in a hearing aid, in sound processing, and for speech in noise.

In a second aspect the present invention relates to a product comprising at least one biomimetic microphone according to the invention, such as a single hearing implant, a mobile device, such as a smartphone, a telecommunication device, and an audio receiver.

In a further aspect the present invention relates to a hearing implant comprising at least one biomimetic microphone according to the invention, being a single hearing implant for transmitting audio input to the brain over one auditory nerve, wherein the biomimetic microphone is adapted to provide output to at least one auditory nerve, such as by a cochlear implant, with the proviso that the hearing implant is adapted to provide output to the at least one auditory nerve at a left side of a human head or at a right side of the human head only. Surprisingly only one, hence a single hearing implant, can be used. The human brain is capable of making use of the output signal of the single hearing implant such that optimal speech in noise perception is more or less achieved. This is considered more effective than inappropriate integration of bilateral applied signals, which results in problems with understanding speech in noisy listening conditions (cocktail party phenomenon).

In yet a further aspect the present invention relates to a method of operating a hearing implant according to the invention, comprising activating the hearing implant, receiving spatial audio input with the at least one first audio receiver and the at least one second audio receiver, processing audio input with the at least one processor, and providing output at one side of the head only to at least one auditory nerve, such as by a cochlear implant, to the brain over one auditory nerve.

The present invention also relates, in a further aspect, to a hearing implant computer program comprising instructions for operating the hearing implant according to the invention, the instructions causing the computer to carry out the following steps: activating the hearing implant, receiving spatial audio input with the at least one first audio receiver and the at least one second audio receiver, processing audio input with the at least one processor, and providing output at one side of the head only to at least one auditory nerve, such as by a cochlear implant, to the brain over one auditory nerve.

Advantages of the present description are detailed throughout the description.

### DETAILED DESCRIPTION

It is noted that examples given, as well as embodiments are not considered to be limiting. The scope of the invention is defined by the claims.

In an exemplary embodiment of the present biomimetic microphone the at least one second audio receiver is selected from a element adapted to rotate comprising said at least one audio receiver eccentric of a rotating axis, that is from a rotating audio receiver and thus moving audio receiver, from a static array of audio receivers located spaced apart from one and another, wherein by addressing individual audio receivers in the static array sound is received at spaced apart locations, wherein in the static array of audio receivers each audio receiver individually is adapted to be addressed by a receiver controller, that is from a static receiver “mimicking” a rotating audio receiver, and a combination thereof, so comprising both examples. FIG. 3 provides an example of the first alternative. FIG. 4 of the second alternative.

In an exemplary embodiment the present biomimetic microphone may comprise at least one actuator for moving said at least one second audio receiver.

In an exemplary embodiment of the present biomimetic microphone the at least one first audio receiver is adapted to operate in pulsating mode, and/or wherein the at least one second audio receiver is adapted to operate in pulsating mode. The term “pulsating” is used to describe that the audio receiver is operated such that it moves rhythmically or vibrates, such as “up and down” in a reciprocal manner.

In an exemplary embodiment of the present biomimetic microphone the biomimetic microphone is adapted to sample sound in phase, to sample sound out of phase, to sample sound in a frequency dependent mode, or a combination thereof. Sound of the audio receivers may be compared to one and another, such as by subtraction. Such may be frequency resolved or not.

In an exemplary embodiment of the present biomimetic microphone the at least one first audio receiver is in a reduced pressure environment, such as a sealed chamber, and/or wherein the at least one second audio receiver is in a reduced pressure environment, such as a sealed chamber. Such does not imply two chambers, only one. The “other side” of the reduced pressure environment, not forming part of the claimed invention, relates to the space having at least one acoustic source, such as the environment of the present microphone, or for that matter, a person wearing the present microphone as a hearing aid.

In an exemplary embodiment of the present biomimetic microphone the reduced pressure environment, each individually, in particular comprise a fluid-to-fluid sound transmitter, such as a membrane.

In the present biomimetic microphone the processor is adapted to select sound in at least one direction, wherein the at least one direction in particular “is away” from the biomimetic microphone, that is “pointing towards” the present microphone.

In an exemplary embodiment of the present biomimetic microphone the processor is adapted to process sound in at least one direction.

In an exemplary embodiment of the present biomimetic microphone the processor is adapted to filter sound, such as sound in a frequency bandwidth, such as noise, and sound from at least one specific direction.

In an exemplary embodiment of the present biomimetic microphone the at least one first audio receiver and at least one second audio receiver are each individually adapted to receive sound in a frequency range of 100 Hz-20 KHz.

In an exemplary embodiment of the present biomimetic microphone the at least one second audio receiver is adapted to receive sound in a cyclic mode with a frequency of 1-100 Hz. That is, reception is with a frequency of 1-100 Hz, that is every 1/100-1 second, whereas the at least one second receiver receives sound in a cyclic mode.

In an exemplary embodiment of the present biomimetic microphone the static array of second audio receivers comprises 1-n second audio receivers, wherein audio receivers are located in a single or multiple curve, such as in circle, or in a spiral, such as an Archimedean spiral, a Fermat's spiral, a logarithmic spiral, a Fibonacci spiral, and a Theodorus spiral, or in a helix, in particular a spiral with 1-5 windings, such as with audio receivers at even or uneven distance from one and another, or a combination thereof.

In an exemplary embodiment of the present biomimetic microphone the static array of second audio receivers comprises 2-210 second audio receivers, in particular 3-28 second audio receivers, such as 4-26 second audio receivers.

In an exemplary embodiment of the present biomimetic microphone first and second audio receivers each individually are selected from transducers, such as a MEMS, a moving coil, a permanent magnet transducer, a balanced armature transducer, and a piezo-element.

In an exemplary embodiment of the present hearing implant the hearing implant is adapted to transfer sound wireless from the biomimetic microphone to the cochlea.

In an exemplary embodiment of the present hearing implant the hearing implant is fully implantable, or wherein the hearing implant comprises an external part, the external part comprising the biomimetic microphone, and in internal part, the internal part comprising at least one of a cochlear implant, and a vibrating implant.

In an exemplary embodiment the present hearing implant may comprise a housing, wherein the housing has a size of 1-5 cm by 1-5 cm and 0.2-2 cm.

In an exemplary embodiment the present hearing implant may comprise at least one coil for wireless transmission.

In an exemplary embodiment of the present hearing implant the implant is adapted to provide a stimulus to the at least one audio nerve, in particular every 1-100 msec, such as every 10-20 msec.

In an exemplary embodiment the present hearing implant may comprise an electro-neuro interface for connecting the hearing implant to the at least one audio nerve, in particular comprising 1-24 electro-neuro interfaces, in particular 9-12 electro-neuro interfaces.

In an exemplary embodiment of the present hearing implant the electro-neuro interphase is adapted to be provided in the cochlea.

The invention is further detailed by the Examples and accompanying figures, which are exemplary and explanatory of nature and are not limiting the scope of the invention. To the person skilled in the art it may be clear that many variants, being obvious or not, may be conceivable falling within the scope of protection, defined by the present claims.

### SUMMARY OF THE FIGURES

FIGS. 1-4 show schematic layouts of exemplary biomimetic microphones.

FIG. 5 shows a schematic layout of reception of sound by a hearing implant including the present biomimetic microphone.

FIG. 6-9 show further experimental results.

### DETAILED DESCRIPTION OF THE FIGURES

In the figures:
1 biomimetic microphone
11 first microphone
12 second microphone
20 processor
30 battery
40 actuator
50 hearing implant
60 cochlear implant

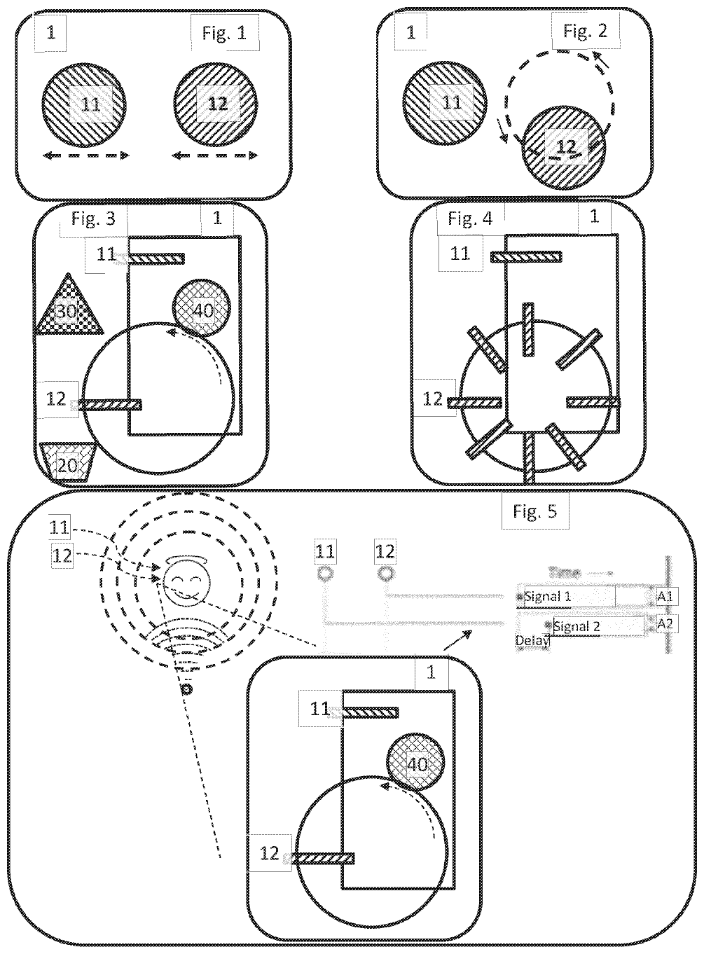

In FIG. 1 a biomimetic microphone 1 is shown, having a first microphone 11 and a second microphone 12. The first microphone 11 and second microphone 12 are adapted to move in a horizontal direction, back and forth, or likewise pulsate in said direction.

In FIG. 2 a biomimetic microphone 1 is shown, having a first microphone 11 and a second microphone 12. The first microphone 11 is static, whereas the second microphone 12 is adapted to move in a circular direction, as indicated by the arrows.

In FIG. 3 a biomimetic microphone 1 is shown, having a first microphone 11 and a second microphone 12. The first microphone 11 is static, whereas the second microphone 12 is adapted to move in a circular direction, as indicated by the arrows. In addition, schematically an actuator 40 for rotating a disc on which microphone 12 is located, a processor 20, and a battery 30 are indicated.

In FIG. 4 a biomimetic microphone 1 is shown, having a first microphone 11 and a series of second microphones 12, in this case 8. The first microphone 11 is static, whereas the second microphones 12 are adapted to be addressed in a circular direction, for instance starting at the most left microphone first, followed by the lower left microphone, the lower middle microphone, the lower right microphone, etc. Any other order of addressing can be chosen.

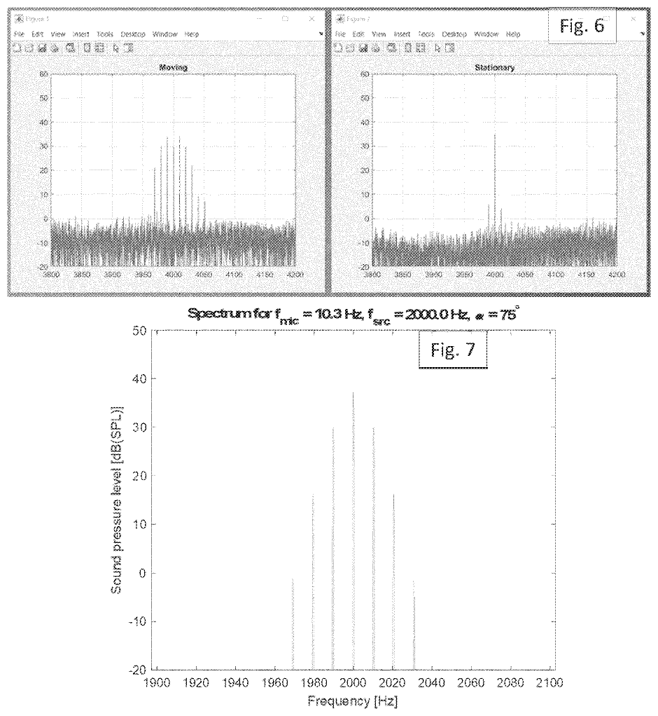

FIG. 5 shows a schematic layout of reception of sound by a hearing implant including the present biomimetic microphone. As microphone 12 moves towards and away from microphone 11 a dynamic time delay is created. Therefore, although the absolute distance between microphone 11 and microphone 12 is limited, a strong direction dependent cue is generated. Also as signal 1 of microphone 12 may be different from a signal 2 of microphone 11, the two signals can easily be discriminated with this biomimetic microphone.

### Experiment

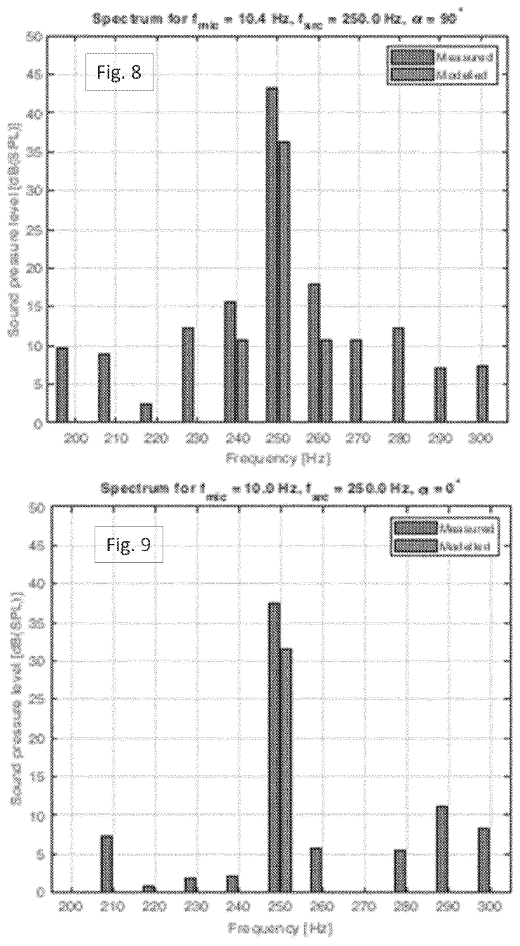

A one-dimensional experiment is performed wherein the present microphone moves in a linear mode towards and from an audio source (left), compared to a situation wherein no movement of the microphone is used (right)(see FIG. 6). The data shows, that even with a single moving microphone, motion in direction to the sound source results in a clearly distinguishable signal. Not shown is that the direction of a sound can also be detected. The harmonics depend on the angle in which the microphone is moved. Harmonics for 0, 15, 45, 75 and 90 degrees for 2000 Hz are found for instance, for which FIG. 7 gives an example for 75 degrees.

It is noted that when testing persons on their ability to detect a direction of a source, consistent results are obtained, in that all direction are detected accurately. Persons with either bilateral conductive hearing loss of unilateral hearing loss, lose the ability to properly detect a direction, irrespective of the sound pressure provided (dB). Surprisingly the present biomimetic microphone provides the above directional detection of sound. In addition, even with only one moving microphone the direction is accurately detected (FIG. 8) except of course when moving along a sound wave (FIG. 9) as in that very specific case no variation in sound pressure can be detected. So the experiments clearly show that the present biomimetic microphone is very well capable of accurately detecting sound in at least one direction, such as by using the processor.

Such makes the present biomimetic microphone in particular suited for a single hearing implant. A person wearing such a hearing implant is now very well capable of detecting a direction of a sound source, and hence, perceiving improved hearing.

1. A biomimetic microphone, the biomimetic microphone comprising
at least two audio receivers, comprising at least one first audio receiver, the at least one first audio receiver being at a first position in the biomimetic microphone, and at least one second audio receiver at a distance from the first position, wherein the at least one second audio receiver is adapted to receive sound in a plane in at least one sequence, wherein the at least one sequence is at least one of continuous and discrete, wherein the plane is selected from a circle, an ellipsoid, a surface section of a sphere, a surface section of a cone, and a surface section of a cylinder,
the combination of the at least one first audio receiver and the at least one second audio receiver adapted to receive spatial audio input,
at least one processor for processing audio input of the at least two audio receivers, and for providing output, wherein the processor is adapted to select sound in at least one direction.

2. The biomimetic microphone according to claim 1, wherein the at least one second audio receiver is selected from an element adapted to rotate said at least one audio receiver eccentric of a rotating axis, from a static array of audio receivers located spaced apart from one and another, wherein by addressing individual audio receivers in the static array sound is received at spaced apart locations, wherein in the static array of audio receivers each audio receiver individually is adapted to be addressed by a receiver controller, and a combination thereof.

3. The biomimetic microphone according to claim 1, when comprising an element adapted to rotate said at least one audio receiver eccentric of a rotating axis, comprising at least one actuator for moving said at least one second audio receiver.

4. The biomimetic microphone according to claim 1, wherein the at least one first audio receiver is adapted to operate in pulsating mode, and wherein the at least one second audio receiver is adapted to operate in pulsating mode, and wherein the biomimetic microphone is adapted to at least one of to sample sound in phase, to sample sound out of phase, to sample sound in a frequency dependent mode, and a combination thereof.

5. (canceled)

6. The biomimetic microphone according to claim 1, wherein the at least one first audio receiver is in a reduced pressure environment, and wherein the at least one second audio receiver is in a reduced pressure environment,
wherein the reduced pressure environment, each individually, comprise a fluid-to-fluid sound transmitter.

7. The biomimetic microphone according to claim 1, wherein the at least one direction is pointing towards from the biomimetic microphone, and/or wherein the processor is adapted to process sound in at least one direction, and wherein the processor is adapted to filter sound, and sound from at least one specific direction.

8. (canceled)

9. The biomimetic microphone according to claim 1, wherein the at least one first audio receiver and at least one second audio receiver are each individually adapted to receive sound in a frequency range of 100 Hz-20 KHz.

10. The biomimetic microphone according to claim 1, wherein the at least one second audio receiver is adapted to receive sound with a frequency of 1-100 Hz.

11. The biomimetic microphone according to claim 2, when comprising the static array of audio receivers located spaced apart from one and another, wherein the static array of second audio receivers comprises 1 to n second audio receivers, wherein audio receivers are located in a curve selected from a single and a multiple curve.

12. The biomimetic microphone according to claim 11, wherein the static array of second audio receivers comprises 2-210 second audio receivers, and wherein first and second audio receivers each individually are selected from transducers, a moving coil, a permanent magnet transducer, a balanced armature transducer, and a piezo-element.

13. (canceled)

14. A product comprising at least one biomimetic microphone according to claim 1.

15. The product of claim 14 being a single hearing implant for transmitting audio input to the brain over one auditory nerve,
wherein the biomimetic microphone is adapted to provide output to at least one auditory nerve,
with the proviso that the hearing implant is adapted to provide output to the at least one auditory nerve at only one of a left side of a human head and at a right side of the human head-only.

16. The hearing implant according to claim 15, wherein the hearing implant is adapted to transfer sound wireless from the biomimetic microphone to the cochlea.

17. The hearing implant according to claim 15, wherein the hearing implant is selected from fully implantable, and from comprising an external part and an internal part, the external part comprising the biomimetic microphone, and the internal part comprising at least one of a cochlear implant, and a vibrating implant.

18. The hearing implant according to claim 15, comprising a housing, wherein the housing has a size of 1-5 cm by 1-5 cm and 0.2-2 cm.

19. The hearing implant according to claim 15, comprising at least one coil for wireless transmission, and wherein the implant is adapted to provide a stimulus to the at least one audio nerve.

20. (canceled)

21. The hearing implant according to claim 15, comprising an electro-neuro interface for connecting the hearing implant to the at least one audio nerve.

22. The hearing implant according to claim 21, wherein the electro-neuro interphase is adapted to be provided in the cochlea.

23. A method of operating a hearing implant the hearing implant comprising a biomimetic microphone, the biomimetic microphone comprising
at least two audio receivers, comprising at least one first audio receiver, the at least one first audio receiver being at a first position in the biomimetic microphone, and at least one second audio receiver at a distance from the first position, wherein the at least one second audio receiver is adapted to receive sound in a plane in at least one sequence, wherein the at least one sequence is at least one of continuous and discrete, wherein the plane is selected from a circle, an ellipsoid, a surface section of a sphere, a surface section of a cone, and a surface section of a cylinder,
the combination of the at least one first audio receiver and the at least one second audio receiver adapted to receive spatial audio input,
at least one processor for processing audio input of the at least two audio receivers, and for providing output, wherein the processor is adapted to select sound in at least one direction;
the method comprising
activating the hearing implant,
receiving spatial audio input with the at least one first audio receiver and the at least one second audio receiver,
processing audio input with the at least one processor, and providing output at one side of the head only to at least one auditory nerve, to the brain over one auditory nerve.

24. A non-transitory computer-readable medium storing a hearing implant computer program comprising instructions for operating a hearing implant, the hearing implant comprising a biomimetic microphone, the biomimetic microphone comprising
at least two audio receivers, comprising at least one first audio receiver, the at least one first audio receiver being at a first position in the biomimetic microphone, and at least one second audio receiver at a distance from the first position, wherein the at least one second audio receiver is adapted to receive sound in a plane in at least one sequence, wherein the at least one sequence is at least one of continuous and discrete, wherein the plane is selected from a circle, an ellipsoid, a surface section of a sphere, a surface section of a cone, and a surface section of a cylinder,
the combination of the at least one first audio receiver and the at least one second audio receiver adapted to receive spatial audio input,
at least one processor for processing audio input of the at least two audio receivers, and for providing output, wherein the processor is adapted to select sound in at least one direction;
the instructions causing the computer to carry out the following steps:
activating the hearing implant,
receiving spatial audio input with the at least one first audio receiver and the at least one second audio receiver,
processing audio input with the at least one processor, and providing output at one side of the head only to at least one auditory nerve, to the brain over one auditory nerve.
